(19) United States
(12) Patent Application Publication
(10) Pub. No.: US 2021/0121673 A1
(43) Pub. Date: Apr. 29, 2021

(54) MICRO-NEEDLE AND METHOD OF MANUFACTURE

(71) Applicant: Gachon University of Industry-Academic Cooperation Foundation, Seongnam-si, Gyeonggi-do (KR)

(72) Inventors: Jung Hwan PARK, Seongnam-si (KR); Won Jun LEE, Seongnam-si (KR); Ji Seok KIM, Seongnam-si (KR); Hye Rin JEONG, Seongnam-si (KR); Jee Hyun PARK, Seoul (KR)

(73) Assignee: Gachon University of Industry-Academic Cooperation Foundation, Seongnam-si, Gyeonggi-do (KR)

(21) Appl. No.: 16/965,177

(22) PCT Filed: Nov. 7, 2018
(86) PCT No.: PCT/KR2018/013452

§ 371 Date: Jan. 5, 2021

(30) Foreign Application Priority Data
Jan. 26, 2018 (KR) 10-2018-0009923

(51) Int. Cl. A61M 37/00

(52) U.S. Cl. CPC A61M 37/0015; A61M 2037/0053

(57) ABSTRACT

Disclosed is a microneedle including: a needle part including a plurality of tips formed from a liquid formulation formed with a medicinal solution and capable of penetrating the skin; and a base for supporting the plurality of tips; and a guide part configured to guide the needle part to penetrate the skin, wherein the needle part is provided with separation guides formed between the tips and the base to separate the tips from the base. According to such a configuration, the tips rapidly penetrate the skin and are rapidly separated therefrom, which allows quantitative drug delivery.

### TECHNICAL FIELD

The present invention relates to a microneedle and a method of manufacturing the same, and more particularly, to a microneedle configured to easily penetrate and be separated such that tips thereof rapidly penetrate into and are separated from an affected area; and a method of manufacturing the same.

### BACKGROUND ART

As general transdermal drug delivery methods, there are passive transdermal drug delivery systems and active transdermal drug delivery systems. Passive transdermal drug delivery systems, which are passive methods depending upon the physicochemical properties of drugs, are characterized by applying a cream, a patch or an ointment to the skin. However, there are limitations in that such passive transdermal drug delivery methods can be applied only when the molecular weight of the drug to be delivered through the skin is 500 Da or less.

To overcome limitation in penetrating the skin in the case of active transdermal drug delivery methods, the stratum corneum having a thickness of 10 μm is physically penetrated with a microneedle to deliver active ingredients. As methods of delivering drugs using a microneedle, there are a method of spreading drug after applying a needle, a method of delivery through a needle surface-coated with a drug, a method of delivery through a molten microneedle containing a drug, and the like.

Ends of such microneedles have a pointed tip shape to penetrate the skin and deliver drugs thereinto. Accordingly, various studies on microneedles are being continuously conducted in recent years so as to increase a drug delivery amount without impeding the penetrating power of microneedles.

### DISCLOSURE

### Technical Problem

Therefore, the present invention has been made in view of the above problems, and it is one object of the present invention to provide a microneedle configured to be easily separated from a base such that tips thereof rapidly penetrate into and are separated from an affected area.

It is another object of the present invention to provide a method of manufacturing a microneedle.

### Technical Solution

In accordance with an aspect of the present invention, the above and other objects can be accomplished by the provision of a microneedle, including: a needle part including a plurality of tips formed from a liquid formulation formed with a medicinal solution and capable of penetrating the skin; and a base for supporting the plurality of tips; and a guide part configured to guide the needle part to penetrate the skin, wherein the needle part is provided with separation guides formed between the tips and the base to separate the tips from the base.

In addition, the guide part may include a plurality of pressing protrusions respectively coupled to the tips; and a support supporting for the plurality of pressing protrusions, wherein the guide part is integrally formed with the needle part, and is separated from the tips after the tips penetrate the skin.

In addition, coupling grooves may be provided at rear ends, coupled to the pressing protrusions, of the tips such that the plurality of pressing protrusions are respectively inserted thereinto, wherein the coupling grooves have a hemispherical shape of being grooved in the penetration direction toward front ends of the tips from rear ends thereof.

In addition, the guide part may be separated from the tips along with the base when the tips penetrate the skin.

In addition, the separation guide may be provided between each of the tips and the base and may be thinner than the tip so that the tips are easily separated from the base due to physical pressing force by the guide part.

In addition, the separation guide may include a plurality of separation holes formed between each of the tips and the base to be spaced apart from each other along the circumference of the tip, and a perforation line may be formed along the plurality of separation holes along which the tips are respectively separated from the base.

In addition, the separation guide may include a plurality of separation holes formed between each of the tips and the base to be spaced apart from each other along a circumference of the coupling groove, and a perforation line may be formed along the plurality of separation holes along which the tips are respectively separated from the base.

In addition, the guide part may include an adhesive band provided on a rear surface of the base with respect to a penetration direction of the needle part, wherein adhesive force between the adhesive band and the skin is greater than adhesive force between the adhesive band and the needle part.

In addition, a hemispherical cavity may be provided in the penetration direction between each of the tips and the adhesive band.

In accordance with another aspect of the present invention, there is provided a method of manufacturing a microneedle, the method including: manufacturing a needle part that includes a plurality of tips formed from a liquid formulation formed with a medicinal solution and a base for supporting the plurality of tips; manufacturing a guide part configured to guide the needle part in a penetration direction in which the needle part penetrates the skin; and coupling the guide part to the needle part such that the needle part is pressed in a direction in which the needle part penetrates the skin, wherein the needle part is provided with a separation guide formed between each of the tips and a base to separate the tip from the base.

In addition, in the manufacturing of the needle part, a polymer solution corresponding to the liquid formulation may be injected into a mold including mold grooves that correspond to the tips having a horn shape wherein a front end is pointed and a diameter increases toward a rear end with respect to a penetration direction in which the needle part penetrates the skin, followed by centrifugation and drying to be molded.

In addition, the separation guide may be provided to have a thinner thickness than each of the tips between the tip and the base, thereby serving to separate the tips from the base due to physical pressing force by the guide part.

In addition, the separation guide may include a plurality of separation holes that are provided to be spaced apart from each other between each of the tips and the base along a circumference of the tip to form a perforation line, and the mold may include a plurality of mold projections that are formed to be spaced apart from each other along the circumferences of the mold grooves to form the separation holes.

In addition, in the manufacturing of the guide part, a plurality of pressing protrusions respectively corresponding to the tips; and a support for supporting the plurality of pressing protrusions may be manufactured by molding, and coupling grooves may be provided in the penetration direction such that the pressing protrusions are respectively inserted and coupled to rear ends of the tips.

In addition, a height of the coupling groove may be adjusted according to a ratio of a volume of the liquid formulation to a volume of the mold for a molding process; and a content of a solid formulation in the liquid formulation.

In addition, the manufacturing of the guide part may include preparing for covering the base; and adhering the adhesive band to a rear surface of the base with respect to a penetration direction of the needle part such that adhesive force between the adhesive band and the skin is greater than adhesive force between the adhesive band and the needle part.

In addition, a hemispherical cavity may be provided between each of the tips and the adhesive band.

In addition, the liquid formulation may be prepared by mixing a biocompatible material with a solvent, the biocompatible material including one or more selected from the group of consisting of hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polyglycolide (PGA), polylactide-glycolide copolymers (PLGA), hyaluronic acid, alginic acid, carrageenan, chondroitin (sulfate), dextran (sulfate), chitosan, polylysine, collagen, gelatin, carboxymethyl chitin, fibrin, agarose, pullulan polyanhydride, polyorthoester, polyetherester, polycaprolactone, polyesteramide, poly(butyric acid), poly(valeric acid), polyurethane, polyacrylate, ethylene-vinyl acetate copolymers, acrylic-substituted cellulose acetate, non-degradable polyurethane, polystyrene, polyvinyl chloride, polyvinyl fluoride, poly(vinyl imidazole), chlorosulphonate polyolefins, polyethylene oxide, polyvinylpyrrolidone (PVP), polyethylene glycol (PEG), polymethacrylate, hydroxypropyl methylcellulose (HPMC), ethyl cellulose (EC), hydroxypropyl cellulose (HPC), carboxymethyl cellulose, cyclodextrin, and copolymers and cellulose of monomers forming the polymers.

In addition, the liquid formulation may be mixed with an additive for increasing mechanical strength, the additive being formed of one or more materials selected from the group consisting of trehalose, glucose, maltose, lactose, lactulose, fructose, turanose, melitose, melezitose, dextran, sorbitol, xylitol, palatinit, mannitol, poly(lactide), poly(glycolide), poly((lactide-co-glycolide), polyanhydride, polyorthoester, polyetherester, polycaprolactone, polyesteramide, poly(butyric acid), poly(valeric acid), polyurethane, polyacrylate, ethylene-vinyl acetate copolymers, acrylic-substituted cellulose acetate, non-degradable polyurethane, polystyrene, polyvinyl chloride, polyvinyl fluoride, poly(vinyl imidazole), chlorosulphonate, polyolefins, polyethylene oxide, polyvinylpyrrolidone (PVP), polyethylene glycol (PEG), polymethacrylate, hydroxypropyl methylcellulose (HPMC), ethyl cellulose (EC), hydroxypropyl cellulose (HPC), carboxymethyl cellulose, cyclodextrin, and copolymers of monomers forming the polymers.

In addition, the liquid formulation may be mixed with an active ingredient, the active ingredient including at least one of a protein/peptide medicine, a hormone, a hormone analogue, an enzyme, an enzyme inhibitor, a signal transduction protein or a portion thereof, an antibody or a portion thereof, a single-chain antibody, a binding protein or a binding domain thereof, an antigen, an adherent protein, a structural protein, a regulatory protein, a toxin protein, a cytokine, a transcription regulator, a blood coagulation factor, and a vaccine.

In addition, the protein/peptide medicine may include at least one of insulin, insulin-like growth factor 1 (IGF-1), growth hormone, erythropoietin, G-granulocyte-colony stimulating factors (CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormone-releasing hormone (LHRH), nafarelin, parathyroid hormone, pramlintide, enfuvirtide (T-20), thymalfasin, and ziconotide.

In addition, the solvent may include inorganic or organic solvents including distilled water (DI water), methanol, ehanol, chloroform dibutyl phthalate, dimethyl phthalate, ethyl lactate, glycerin), isopropyl alcohol, lactic acid, and propylene glycol.

### Advantageous Effects

According to the present invention having the configuration, first, a guide part is provided to guide tips of a needle part in a direction of skin penetration serves the tips, thereby being capable of rapidly penetrating an affected area and being rapidly separated therefrom.

Second, a separation guide is provided to have a relatively thin thickness compared to the tips, thereby being capable of rapidly separating the tips from a base by physical pressing force and, accordingly, increasing the penetration power.

Third, a plurality of separation holes are formed along the circumference of each of the tips so that a kind of perforation line is formed between the tip and the base. Accordingly, cracks due to stress concentration can be induced even by relatively small pressure, so that the tips can be rapidly separated from the base.

Fourth, a separation guide is provided between each of the tips and the base so that the tip can be rapidly separated from the base. Accordingly, it is possible to quantitatively supply a medicine contained in the tips, which can contribute to improving patient reliability.

Fifth, the base can be easily separated from the tips penetrated into the skin due to a simple operation wherein an adhesive band is integrated with to a needle part and then is attached to the skin, followed by removing the attached adhesive band.

Sixth, the lengths of pressing protrusions and the heights of coupling grooves coupled to the pressing protrusions can be adjusted to correspond to various drugs and dosage conditions.

### DESCRIPTION OF DRAWINGS

FIG. 1 is a sectional view schematically illustrating a microneedle according to a preferred first embodiment of the present invention.

FIG. 2 is a sectional view schematically illustrating a needle part illustrated in FIG. 1.

FIG. 3 illustrates images of tips of the needle part, illustrated in FIG. 2, schematically photographed with an optical microscope.

FIG. 4 illustrates images of the needle part, illustrated in FIG. 2, schematically photographed with a scanning electron microscope.

FIG. 5 illustrates an image of a guide part, illustrated in FIG. 1, schematically photographed with an optical microscope.

FIGS. 6A to 6D sequentially illustrate an operation in which the microneedle illustrated in FIG. 1 penetrates the skin.

FIG. 7 is a perspective view schematically illustrating a microneedle according to a preferred second embodiment of the present invention.

FIGS. 8A to 8E schematically illustrate a step-by-step method of manufacturing the microneedle illustrated in FIG. 7.

FIGS. 9A to 9C illustrate images of a needle part of the microneedle according to the second embodiment schematically photographed with a scanning electron microscope.

FIG. 10 is a perspective view schematically illustrating a microneedle according to a preferred third embodiment of the present invention.

FIGS. 11A to 11D sequentially illustrate an operation in which the microneedle illustrated in FIG. 10 penetrates the skin.

### BEST MODE

Now, a preferred fist embodiment of the present invention will be described with reference to the accompanying drawings.

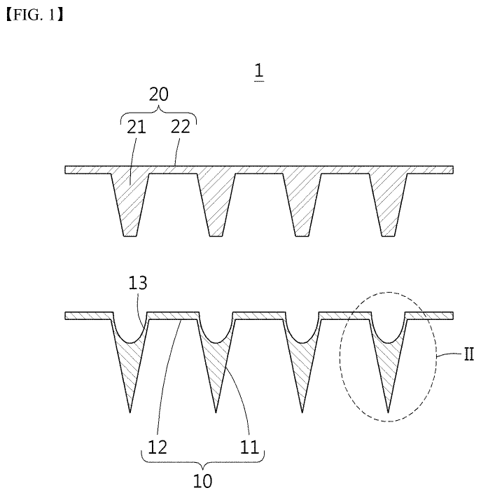

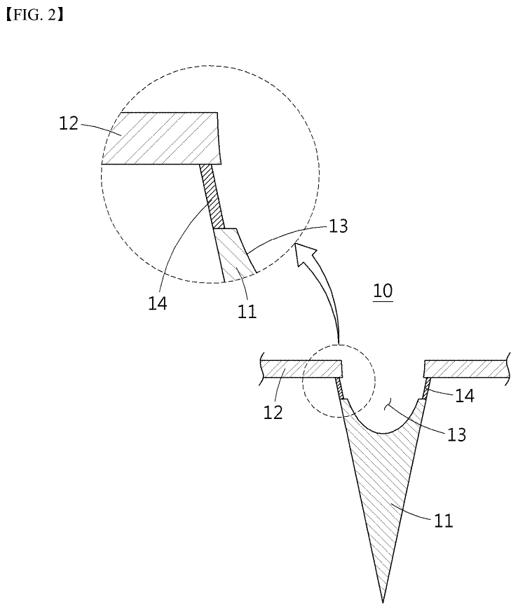

Referring to FIG. 1, a microneedle 1 according to a preferred first embodiment of the present invention includes a needle part 10 and a guide part 20.

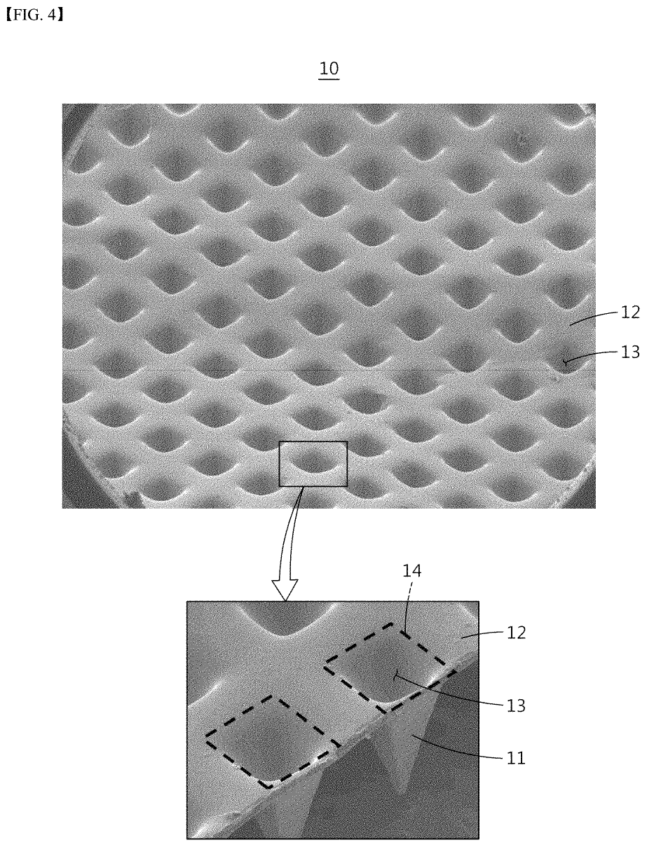

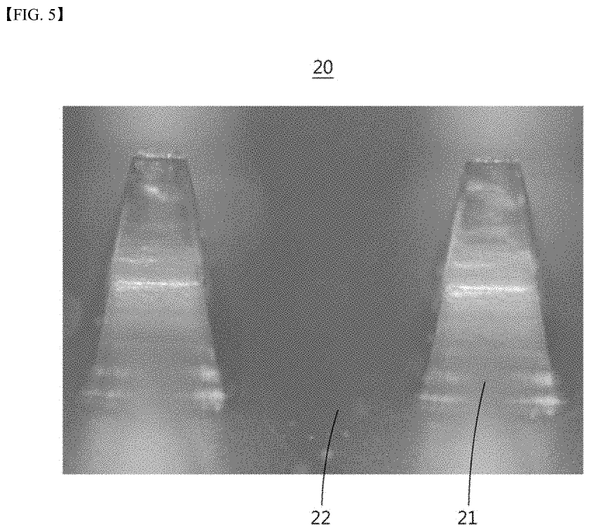

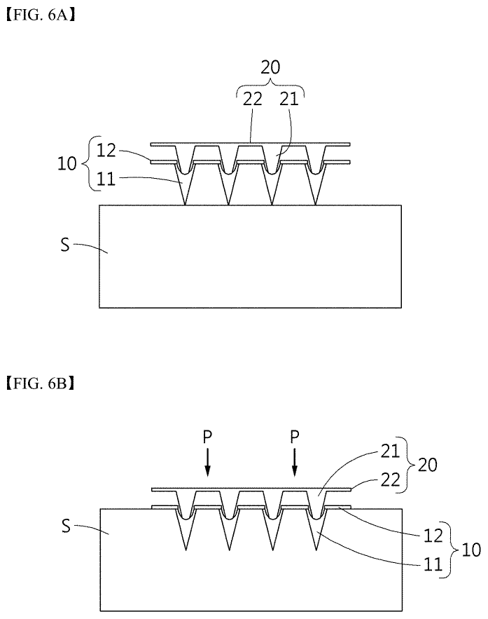

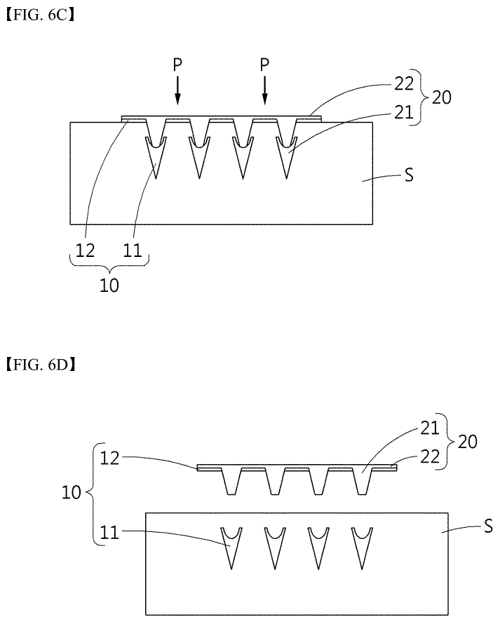

The needle part 10 includes a plurality of tips 11 formed from a liquid formulation formed with a medicinal solution and capable of penetrating the skin S (see FIG. 6); and a base 12 for supporting the plurality of tips 11. The needle part 10 is a body of the microneedle 1 for supplying the liquid formulation to the skin S.

The plurality of tips 11 are simultaneously supported by the base 12. The base 12 is preferably formed of a flexible material to be in close contact with the skin S.

Meanwhile, a separation guide 14 thinner than the tip 11 is provided between each of the tips 11 and the base 12. The thickness of the separation guide 14 is relatively, greatly reduced compared to that of the tips 11 to be easily broken by external impact.

The separation guide 14 may be formed between the tip 11 and the base 12 along the circumference of the tip 11. Stress is concentrated on an edge of the tip 11 as the separation guide 14 is formed along the circumference of the tip 11, so that the edge is cracked even by relatively small pressure and, accordingly, the tip 11 is easily separated from the base 12.

In addition, the plurality of tips 11 have a horn shape wherein, with respect to the penetration direction P, a front end of the tip 11 is pointed and the diameter of the tip 11 gradually increases toward a rear end of the tip 11 (see FIG. 6). A coupling groove 13, which is formed in the penetration direction P, is provided at a rear end of each of the plurality of tips to be coupled with the guide part 20 described below. More particularly, the coupling groove 13 is formed in a hemispherical shape characterized by being grooved from the rear end of the tips 11 to the front end thereof, and the separation guide 14 may be provided along a side of the coupling groove 13 of the tip 11. Here, the height of the coupling groove 13, i.e., the depth thereof, may be adjusted depending upon a drug amount, a penetration condition, or the like of the liquid formulation as shown in FIG. 3.

The thickness of the separation guide 14 and the height of the coupling groove 13 may be adjusted according to the content of a molding solution to the volume of a mold used in a molding process of manufacturing the tips 11, i.e., volume of a molding solution and a content of a solid formulation in a molding solution. Here, the solid formulation may include a thickener, surfactant, stabilizer and active ingredients to be added to a molding solution. In addition, formation of the separation guide 14 and the coupling groove 13 may be related to interfacial interactions between a mold for manufacturing the microneedle 1 and the molding solution.

For reference, in the present embodiment, a mold having a surface tension of 10 to 40 mN/m may be used to manufacture the microneedle 1, and PDMS, which is hydrophobic, may have a surface tension of 22 to 23 mN/m. In addition, a molding solution for manufacturing the dissolvable microneedle 1 is prepared based on water, and spreading of the molding solution may be adjusted by using a surfactant. Accordingly, spreading of the solution for manufacturing the soluble microneedle 1 may be adjusted by the viscosity of the solution, so that the thickness of the molding solution may be locally controlled in the mold.

Here, the viscosity of the solution for manufacturing the microneedle 1 may be controlled by adjusting the concentrations of a thickener, a surfactant, a stabilizer and active ingredients. In addition, the solution for manufacturing the microneedle 1 may be relatively thin, compared to other regions, in a region where the separation guide 14 is to be formed.

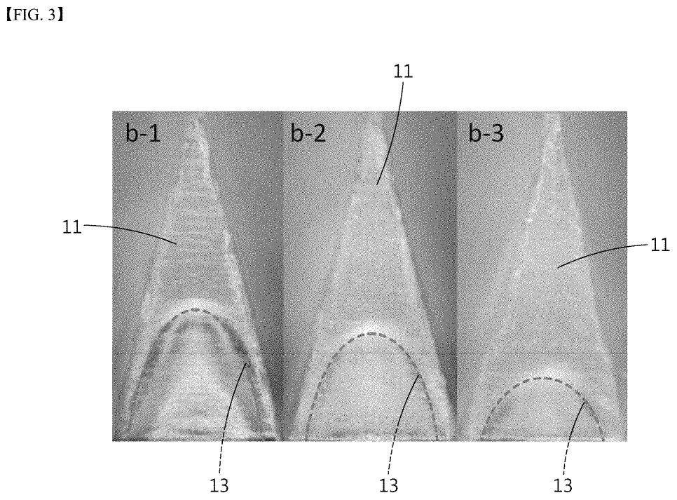

FIG. 4 illustrates an image of the needle part 10 photographed with a scanning electron microscope. As shown in FIG. 4, each of the plurality of tips 11 is provided with the coupling groove 13 and is supported by the base 12. Here, as in the enlarged view shown in FIG. 4, the coupling groove 13 is provided to have a predetermined depth toward a pointed front end of the tips 11 from the rear end thereof, and the separation guide 14 is relatively, thinly formed along the circumference of the coupling groove 13.

The guide part 20 is a kind of pressing pusher for pressing the needle part 10 into the skin S in a penetration direction. The guide part 20 includes a plurality of pressing protrusions 21 respectively coupled to the plurality of tips 11; and a support 22 for supporting the plurality of pressing protrusions 21.

The pressing protrusions 21 have a height that can be inserted into the coupling groove 13 formed at a rear end of the tip 11, and protrude from the support 22. Here, the pressing protrusions 21 are respectively inserted into the coupling grooves 13 and are integrally coupled with the needle part 10. The pressing protrusion 21 may apply physical impact to the separation guide 14 formed along the circumference of the coupling groove 13. The guide part 20 provided with the pressing protrusions 21 is manufactured using the same mold as that in the needle part 10. In particular, polylactic acid (PLLA) was melted at about 195° C. and then applied to the mold to manufacture the guide part 20.

Here, the pressing protrusions 21 may protrude to have a height of 650 μm from the support 22 as shown in FIG. 5 and may have a bottom length of about 370 μm. In addition, the support 22 may have a diameter of about 1 cm and may support about 95 to 100 pressing protrusions 21.

For reference, the height of the pressing protrusions 21 may also be adjusted depending upon the height of the coupling grooves 13, a penetration condition, or the like.

An operation of penetrating the microneedle 1 according to the first embodiment of the present invention having the aforementioned configuration into the skin S is described with reference to FIG. 6.

As shown in FIGS. 6A and 6B, the needle part 10, in a state of being integrally coupled with the guide part 20, penetrates the skin S. Here, the guide part 20 presses the needle part 10 in the penetration direction P as shown in FIGS. 6B and 6C, particularly presses the tips 11 of the needle part 10 in the penetration direction P in the state that the pressing protrusions 21 are inserted into the coupling grooves 13 as shown in FIG. 6C. Here, the needle part 10 may be applied for 10 seconds with a force of about 10 N by the guide part 20.

Accordingly, the separation guides 14 included in the plurality of tips 11 are easily cracked by physical pressing force of the pressing protrusion 21, so that the tips 11 are separated from the base 12 and completely penetrate the skin S, as shown in FIG. 6C. Next, the guide part 20 is separated along with the base 12 from the skin S, so that the tips 11 remaining in the skin S are finally melted to provide a drug to the skin S, as shown in FIG. 6D.

A method of manufacturing the microneedle 1 according to the present invention having the aforementioned configuration is described with reference to FIG. 1.

As shown in FIG. 1, the plurality of tips 11 of the needle part 10 are manufactured to be supported by the base 12. In addition, the guide part 20 is provided with the plurality of pressing protrusions 21 that respectively correspond to the plurality of tips 11.

Hereinafter, a method of manufacturing the needle part 10 is described in detail.

First, a viscous agent is mixed with distilled water (DW) to prepare a molding solution including the liquid formulation for manufacturing the needle part 10. Here, hyaluronic acid is used as a viscous agent, Lutrol f68 is used as a surfactant, and Calcein is used as a model drug.

Next, the prepared molding solution is applied to a mold corresponding to the shape of the needle part 10, and then a centrifugation process is performed at 3500 rpm and 25° C. for 10 min, followed by drying in a humidity chamber. The needle part 10 may be manufactured in a single process by a mold casting method.

Here, the composition of the molding solution, i.e., the liquid formulation, supplied to the mold is exemplified as including 3.5% of hyaluronic acid (HA) as a viscous agent, 0.035% of Lutrol f68 as a surfactant, 0.7% of calcein as a model drug, and 95.765% of distilled water. 0.4 g of the molding solution prepared in a such a manner is cast into a mold. A finally produced solid formulation, i.e., the microneedle 1, may have a mass of about 0.017 g.

Meanwhile, the amount of the molding solution loaded into the mold may be controlled to adjust the thickness of the separation guide 14 and the sizes of the coupling grooves 13 which are included in the tips 11 of the needle part 10. For reference, loading amounts applied to the tips 11 shown in FIG. 3 may be about 0.3 g in b-1, 0.35 g in b-2, and 0.4 g in b-3, and a final mass of the needle part 10 may be about 0.013 gin b-1, 0.015 g in b-2, and 0.017 g in b-3.

The molded needle part 10 and guide part 20 are integrally coupled with each other. That is, the pressing protrusion 21 is inserted and coupled with the coupling groove 13 of each of the tips 11, thereby producing a final product.

Meanwhile, a drug used to manufacture the needle part 10 is mixed with a biocompatible material. The biocompatible material may include, as well as the exemplified hyaluronic acid, one or more selected from the group of consisting of hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polyglycolide (PGA), polylactide-glycolide copolymers (PLGA), hyaluronic acid, alginic acid, carrageenan, chondroitin (sulfate), dextran (sulfate), chitosan, polylysine, collagen, gelatin, carboxymethyl chitin, fibrin, agarose, pullulan polyanhydride, polyorthoester, polyetherester, polycaprolactone, polyesteramide, poly(butyric acid), poly(valeric acid), polyurethane, polyacrylate, ethylene-vinyl acetate copolymers, acrylic-substituted cellulose acetate, non-degradable polyurethane, polystyrene, polyvinyl chloride, polyvinyl fluoride, poly(vinyl imidazole), chlorosulphonate polyolefins, polyethylene oxide, polyvinylpyrrolidone (PVP), polyethylene glycol (PEG), polymethacrylate, hydroxypropyl methylcellulose (HPMC), ethyl cellulose (EC), hydroxypropyl cellulose (HPC), carboxymethyl cellulose, cyclodextrin, and copolymers and cellulose of monomers forming the polymers.

In addition, the liquid formulation is formed by mixing the biocompatible material with an additive for increasing mechanical strength. The additive is formed of one or more materials selected from the group consisting of trehalose, glucose, maltose, lactose, lactulose, fructose, turanose, melitose, melezitose, dextran, sorbitol, xylitol, palatinit, mannitol, poly(lactide), poly(glycolide), poly((lactide-co-glycolide), polyanhydride, polyorthoester, polyetherester, polycaprolactone, polyesteramide, poly(butyric acid), poly(valeric acid), polyurethane, polyacrylate, ethylene-vinyl acetate copolymers, acrylic-substituted cellulose acetate, non-degradable polyurethane, polystyrene, polyvinyl chloride, polyvinyl fluoride, poly(vinyl imidazole), chlorosulphonate, polyolefins, polyethylene oxide, polyvinylpyrrolidone (PVP), polyethylene glycol (PEG), polymethacrylate, hydroxypropyl methylcellulose (HPMC), ethyl cellulose (EC), hydroxypropyl cellulose (HPC), carboxymethyl cellulose, cyclodextrin, and copolymers of monomers forming the polymers.

In addition, the liquid formulation is formed by mixing the biocompatible material with an active ingredient. The active ingredient includes a protein/peptide medicine without being limited thereto. The active ingredient may include at least one of a protein/peptide medicine, a hormone, a hormone analogue, an enzyme, an enzyme inhibitor, a signal transduction protein or a portion thereof, an antibody or a portion thereof, a single-chain antibody, a binding protein or a binding domain thereof, an antigen, an adherent protein, a structural protein, a regulatory protein, a toxin protein, a cytokine, a transcription regulator, a blood coagulation factor, and a vaccine. More particularly, the protein/peptide medicine includes at least one of insulin, insulin-like growth factor 1 (IGF-1), growth hormone, erythropoietin, G-granulocyte-colony stimulating factors (CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormone-releasing hormone (LHRH), nafarelin, parathyroid hormone, pramlintide, enfuvirtide (T-20), thymalfasin, and ziconotide.

In addition, the solvent included in the liquid formulation serves to dissolve the biocompatible material. The solvent includes at least one of inorganic or organic solvents including distilled water (DI water), methanol, ehanol, chloroform dibutyl phthalate, dimethyl phthalate, ethyl lactate, glycerin), isopropyl alcohol, lactic acid, and propylene glycol.

FIG. 7 illustrates a microneedle 100 according to a preferred second embodiment of the present invention.

As shown in FIG. 7, the microneedle 100 according to the second embodiment includes a needle part 110 and a guide part 120.

The needle part 110 includes a plurality of tips 111 formed from a liquid formulation formed with a medicinal solution and capable of penetrating the skin; and a base 112 for supporting the plurality of tips 111. Here, the needle part 110 includes separation guides 114 provided between the plurality of tips 111 and the base 112 and configured to separate the plurality of tips 111 from the base 112.

The separation guides 114 according to the second embodiment include a plurality of separation holes 114 that are formed between each of the plurality of tips 111 and the base 112 and are spaced from each other along the circumference of the tip 111. Hereinafter, the separation guides 114 are referred to as the separation holes 114 for convenience of description.

The plurality of separation holes 114 serve to form a perforation line along the circumference of a rear end of the tip 111, thereby inducing cracks between the tip 111 and the base 112 even under slight pressure.

The guide part 120 includes a plurality of pressing protrusions 121 respectively coupled to the plurality of tips 111; and a support 122 for supporting the plurality of pressing protrusions 121. Here, each of the plurality of pressing protrusions 121 is inserted and coupled to a coupling groove 113 formed at a rear end of the tip 111, and the plurality of separation holes 114, as a separation guide, are formed along the circumference of the coupling groove 113 to provide a perforation line.

Since the configuration of the guide part 120 is similar to that of the first embodiment described with reference to FIG. 1, detailed descriptions thereof are omitted.

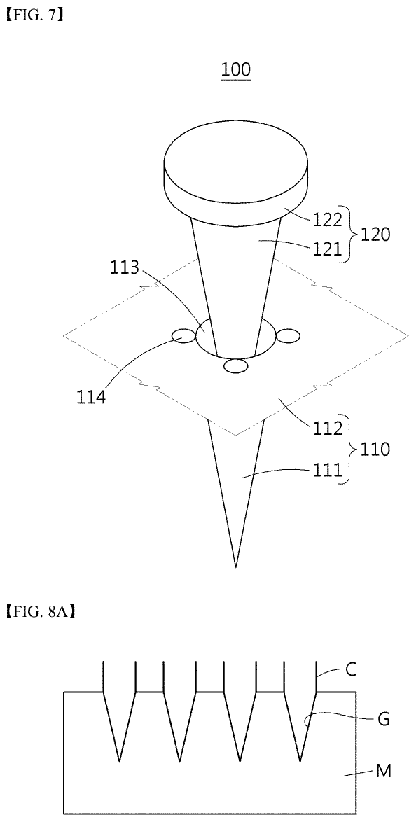

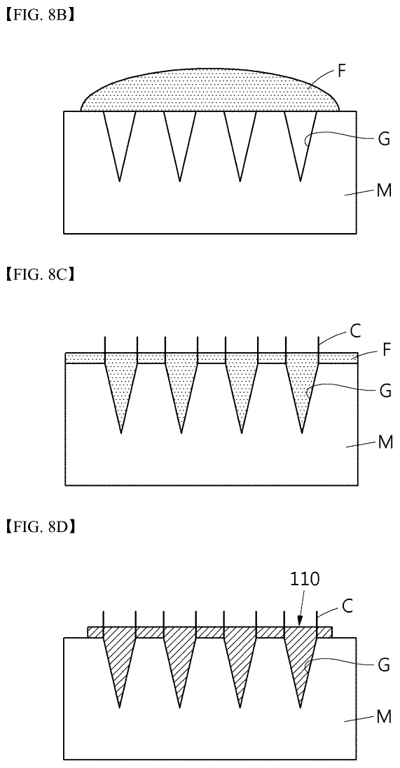

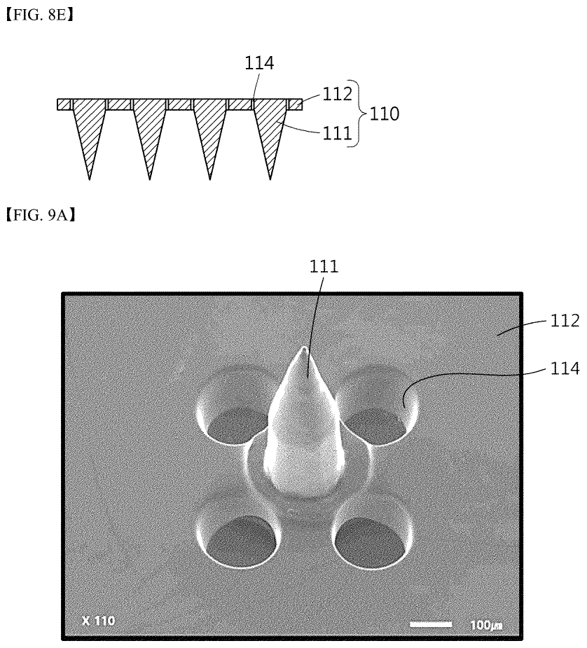

FIGS. 8A to 8E sequentially illustrate a method of manufacturing the microneedle 100 according to the second embodiment. Here, FIGS. 8A to 8E only illustrate the needle part 110 of the microneedle 100 according to the second embodiment and do not illustrate and describe a method of manufacturing the guide part 120 because the guide part 120 is the same as that of the first embodiment.

As shown in FIG. 8A, a mold M is prepared to correspond to the needle part 110 according to the second embodiment. Here, the mold M includes mold grooves G having a conical shape to correspond to the plurality of tips 111. Here, the mold grooves G may have a polygonal pyramid shape, such as a square pyramid or a pentagonal pyramid, instead of a conical shape, and the shape of the tips 111 may be changed according to the shape of the mold grooves G.

In addition, the mold M is provided with a plurality of mold projections C to form separation holes 114 as a separation guide. The plurality of mold projections C are provided to be spaced apart from each other by a predetermined distance along the circumference of each of the mold grooves G although they are not illustrated in detail.

When the mold M including the mold grooves G and the mold projections C is prepared, a polymer solution F is injected into the mold grooves G of the mold M as shown in FIG. 8B. Here, the polymer solution F is injected in an amount of about 0.5 g to manufacture the needle part 110. In the present embodiment, the polymer solution F is exemplified as including 5% of carboxymethyl cellulose (carboxy methyl cellulose sodium) as a biocompatible material, 15% of sucrose, and 80% of distilled water (DW). The polymer solution F is injected in a sufficient amount to cover the mold projections C.

For reference, the polymer solution F, i.e., a drug for forming the needle part 110, is not limited to the described particulars and may include the various drug components described in the first embodiment.

When the polymer solution F is injected into the mold M, the mold M is cast as shown in FIG. 8C. In the present embodiment, the mold casting is exemplified as being performed 6 times for 5 minutes each at a speed of 3000 rpm.

The polymer solution F is dried in the mold-cast state as shown in FIG. 8D. Here, the drying is performed at about 25° C. for about 12 hours until about 24% is dried, thereby producing the needle part 110. The manufactured needle part 110 is separated from the mold M as shown in FIG. 8E, thereby finally producing the needle part 110.

Meanwhile, the needle part 110 shown in FIGS. 8A to 8E does not include coupling grooves 113 to be coupled with the guide parts 120, but the coupling grooves 113 may be formed in a centrifugation process shown in FIG. 8C.

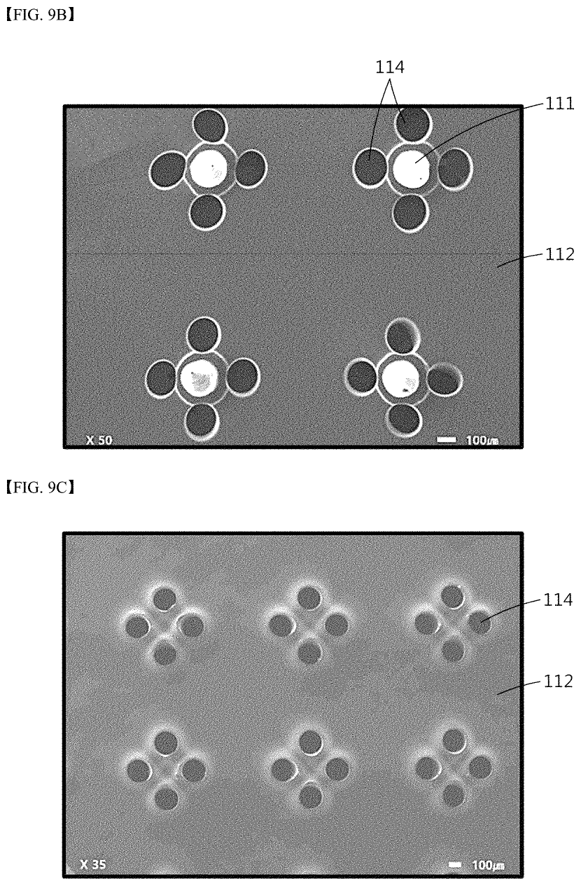

The needle part 110 manufactured according to the method shown in FIGS. 8A to 8E was photographed with a scanning electron microscope (SEM), and images thereof are illustrated in FIGS. 9A to 9C.

FIG. 9A is a photograph showing a state where the separation holes 114 are formed at the base 112 along the circumference of the tip 111, and FIGS. 9B and 9C are respectively top and bottom photographs showing the state where the separation holes 114 penetrate the base 112. As shown in FIGS. 9A to 9C, the separation holes 114 are formed to be spaced apart from each other along the circumference of a rear end, which is connected to the base 112, of the tip 111, not a front end of the tip 111. By the separation holes 114, a perforation line is formed between the tip 111 and the base 112, so that cracks are generated due to shear stress generation along the circumference of the tip 111 even under slight pressure provided by the guide part 120 and, accordingly, the tip 111 is easily separated.

For reference, although the coupling grooves 13 and 113 are illustrated and exemplified as being formed at rear ends of the tips 11 and 111 in the first and second embodiments, the present invention is not limited thereto. In particular, when the base 112 is provided with the plurality of separation holes 114 for forming a kind of perforation line as in the second embodiment, the pressing protrusions 121 may cause sufficient cracks in the base 112 and separation even with a small force although the coupling grooves 113, as a kind of cavities, are not provided at a rear end of the tip 111. That is, the tip 111 may be easily separated from the base 112 by the separation holes 114 even when a small pressing force is applied to the pressing protrusions 121 without a separate guide such as cavities.

FIG. 10 schematically illustrates a microneedle 200 according to a third embodiment of the present invention.

As shown in FIG. 10, the microneedle 200 according to the third embodiment includes a needle part 210 and a guide part 220.

The needle part 210 includes a plurality of tips 211 and a base 212 for supporting the plurality of tips 211. The needle part 210 includes a plurality of separation holes 214 formed along the circumference of the tip 211 to form a perforation line between each of the tips 211 and the base 212 as in the second embodiment.

Since the configuration of the needle part 210 is similar to that of the second embodiment described above, a detailed description thereof is omitted. In addition, since a method of manufacturing the needle part 210 is also similar to that of the second embodiment described with reference to FIGS. 8A to 8E, a detailed description thereof is omitted.

The guide part 220 includes an adhesive band provided on a rear surface of the base 212 with respect to a penetration direction of the needle part 210. Hereinafter, the guide part 220 according to the third embodiment is referred to as an adhesive band 220 for convenience of description.

The adhesive band 220 is provided to have an area that can sufficiently cover the base 212, and is provided on a rear surface of the needle part 210 such that adhesive force to the needle part 210 is less than adhesive force to the skin S. That is, adhesive force between the adhesive band 220 and the base 212 is greater than adhesive force between the adhesive band 220 and the skin S. Accordingly, even if the adhesive band 220 is removed after the microneedle 200 penetrates the skin S, the tip 211 remains in the skin S.

For reference, hemispherical cavities 213 are provided between the plurality of tips 211 and the adhesive band 220. The cavities 213 may facilitate the separation of the tips 213 even with a small pressing force applied to the adhesive band 220.

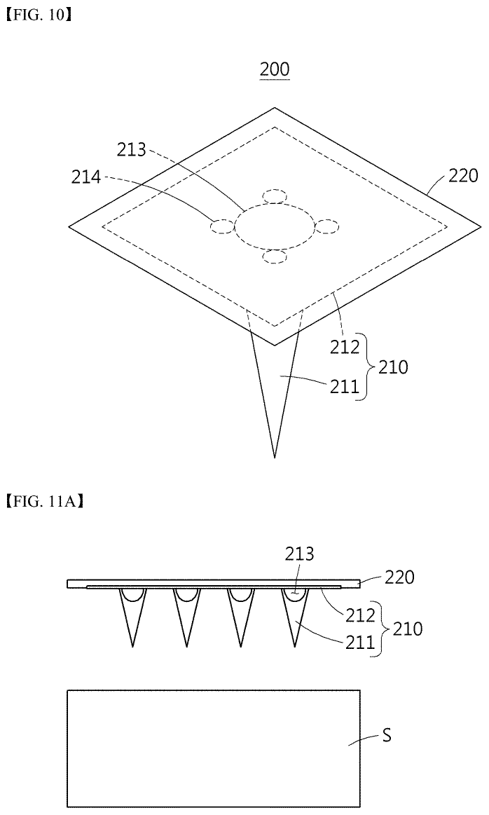

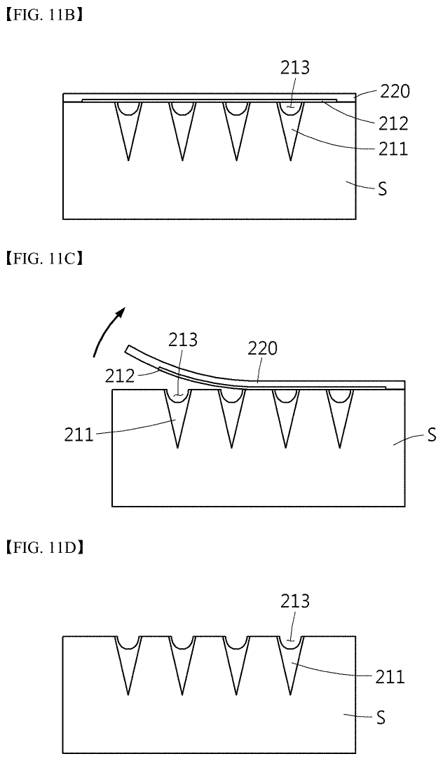

An operation of penetrating the skin S with the microneedle 200 according to the third embodiment is described with reference to FIGS. 11A to 11D.

As shown in FIGS. 11A and 11B, the microneedle 200 according to the third embodiment in a state in which the needle part 210 thereof is adhered and integrated with the adhesive band 220 is applied into the skin S. The adhesive band 220 is separated in a state in which the needle part 210 completely penetrates the skin S as in FIG. 11C.

Here, stress concentration occurs in an edge region of the tip 211 even by a small pressing force due to the plurality of separation holes 214 and the cavity 213 that form a perforation line between the base 212 and the tip 211, so that the tip 211 can be easily separated from the base 212. In addition, since adhesive force of the adhesive band 220 to the skin S is relatively great, the tip 211 is guided to be separated from the base 212 when the adhesive band 220 is separated from the skin S.

The tips 211 separated from the base 212 remain in the skin S as in FIG. 11D, so that the tips 211 sufficiently penetrate the skin S and, accordingly, a quantitative amount of drug can be supplied.

For reference, although FIG. 10 illustrates and exemplifies the needle part 210 of the microneedle 200 according to the third embodiment as including the cavity 213, the present invention is not limited thereto. That is, due to a perforation line, i.e., a separation guide, formed by the plurality of separation holes 214, pressing force applied to the adhesive band 220 may sufficiently cause crack generation between the tips 211 and the base 212 to be separated from the skin S.

In addition, although the cavities 113 and 213 of the second and third embodiments are exemplified as having circular shapes and including 4 holes formed along the circumferences of the tips 111 and 211, the shapes and numbers of the cavities 113 and 213 are not limited thereto.

In addition, although the plurality of separation holes 113 and 213 of the microneedles 100 and 200 of the second and third embodiments have been described as being formed by molding processes, the present invention is not limited thereto. That is, it is natural that the cavities 113 and 213, as perforation lines, may be formed between the tips 111 and 211 and the bases 112 and 212 by various modified methods such as a physical method using laser cutting, a knife, or the like.

While the present invention has been described referring to the preferred embodiments, those skilled in the art will appreciate that many modifications and changes can be made to the present invention without departing from the spirit and essential characteristics of the present invention.

1. A microneedle, comprising:
a needle part comprising a plurality of tips formed from a liquid formulation formed with a medicinal solution and capable of penetrating the skin; and a base for supporting the plurality of tips; and
a guide part configured to guide the needle part to penetrate the skin,
wherein the needle part is provided with separation guides formed between the tips and the base to separate the tips from the base.

2. The microneedle according to claim 1, wherein the guide part comprises a plurality of pressing protrusions respectively coupled to the tips; and a support supporting for the plurality of pressing protrusions,
wherein the guide part is integrally formed with the needle part, and is separated from the tips after the tips penetrate the skin.

3. The microneedle according to claim 2, wherein coupling grooves are provided at rear ends, coupled to the pressing protrusions, of the tips such that the plurality of pressing protrusions are respectively inserted thereinto,
wherein the coupling grooves have a hemispherical shape of being grooved in the penetration direction toward front ends of the tips from rear ends thereof.

4. The microneedle according to claim 1, wherein the guide part is separated from the tips along with the base when the tips penetrate the skin.

5. The microneedle according to claim 1, wherein the separation guide is provided between each of the tips and the base and is thinner than the tip so that the tips are easily separated from the base due to physical pressing force by the guide part.

6. The microneedle according to claim 1, wherein the separation guide comprises a plurality of separation holes formed between each of the tips and the base to be spaced apart from each other along the circumference of the tip, and a perforation line is formed along the plurality of separation holes along which the tips are respectively separated from the base.

7. The microneedle according to claim 3, wherein the separation guide comprises a plurality of separation holes formed between each of the tips and the base to be spaced apart from each other along a circumference of the coupling groove, and a perforation line is formed along the plurality of separation holes along which the tips are respectively separated from the base.

8. The microneedle according to claim 1, wherein the guide part comprises an adhesive band provided on a rear surface of the base with respect to a penetration direction of the needle part, wherein adhesive force between the adhesive band and the skin is greater than adhesive force between the adhesive band and the needle part.

9. The microneedle according to claim 8, wherein a hemispherical cavity is provided in the penetration direction between each of the tips and the adhesive band.

10. A method of manufacturing a microneedle, the method comprising:
manufacturing a needle part that comprises a plurality of tips formed from a liquid formulation formed with a medicinal solution and a base for supporting the plurality of tips;
manufacturing a guide part configured to guide the needle part in a penetration direction in which the needle part penetrates the skin; and
coupling the guide part to the needle part such that the needle part is pressed in a direction in which the needle part penetrates the skin,
wherein the needle part is provided with a separation guide formed between each of the tips and a base to separate the tip from the base.

11. The method according to claim 10, wherein, in the manufacturing of the needle part, a polymer solution corresponding to the liquid formulation is injected into a mold comprising mold grooves that correspond to the tips having a horn shape wherein a front end is pointed and a diameter increases toward a rear end with respect to a penetration direction in which the needle part penetrates the skin, followed by centrifugation and drying to be molded.

12. The method according to claim 11, wherein the separation guide is provided to have a thinner thickness than each of the tips between the tip and the base, thereby serving to separate the tips from the base due to physical pressing force by the guide part.

13. The method according to claim 11, wherein the separation guide comprises a plurality of separation holes that are provided to be spaced apart from each other between each of the tips and the base along a circumference of the tip to form a perforation line, and the mold comprises a plurality of mold projections that are formed to be spaced apart from each other along the circumferences of the mold grooves to form the separation holes.

14. The method according to claim 10, wherein, in the manufacturing of the guide part, a plurality of pressing protrusions respectively corresponding to the tips; and a support for supporting the plurality of pressing protrusions are manufactured by molding, and coupling grooves are provided in the penetration direction such that the pressing protrusions are respectively inserted and coupled to rear ends of the tips.

15. The method according to claim 14, wherein a height of the coupling groove is adjusted according to a ratio of a volume of the liquid formulation to a volume of the mold for a molding process; and a content of a solid formulation in the liquid formulation.

16. The method according to claim 10, wherein the manufacturing of the guide part comprises:
preparing for covering the base; and
adhering the adhesive band to a rear surface of the base with respect to a penetration direction of the needle part such that adhesive force between the adhesive band and the skin is greater than adhesive force between the adhesive band and the needle part.

17. The method according to claim 16, wherein a hemispherical cavity is provided between each of the tips and the adhesive band.

18. The method according to claim 10, wherein the liquid formulation is prepared by mixing a biocompatible material with a solvent, the biocompatible material comprising one or more selected from the group of consisting of hyaluronic acid, alginic acid, pectin, carrageenan, chondroitin (sulfate), dextran (sulfate), chitosan, polylysine, collagen, gelatin, carboxymethyl chitin, fibrin, agarose, pullulan polylactide, polyglycolide (PGA), polylactide-glycolide copolymers (PLGA), hyaluronic acid, alginic acid, carrageenan, chondroitin (sulfate), dextran (sulfate), chitosan, polylysine, collagen, gelatin, carboxymethyl chitin, fibrin, agarose, pullulan polyanhydride, polyorthoester, polyetherester, polycaprolactone, polyesteramide, poly(butyric acid), poly(valeric acid), polyurethane, polyacrylate, ethylene-vinyl acetate copolymers, acrylic-substituted cellulose acetate, non-degradable polyurethane, polystyrene, polyvinyl chloride, polyvinyl fluoride, poly(vinyl imidazole), chlorosulphonate polyolefins, polyethylene oxide, polyvinylpyrrolidone (PVP), polyethylene glycol (PEG), polymethacrylate, hydroxypropyl methylcellulose (HPMC), ethyl cellulose (EC), hydroxypropyl cellulose (HPC), carboxymethyl cellulose, cyclodextrin, and copolymers and cellulose of monomers forming the polymers.

19. The method according to claim 10, wherein the liquid formulation is mixed with an additive for increasing mechanical strength, the additive being formed of one or more materials selected from the group consisting of trehalose, glucose, maltose, lactose, lactulose, fructose, turanose, melitose, melezitose, dextran, sorbitol, xylitol, palatinit, mannitol, poly(lactide), poly(glycolide), poly((lactide-co-glycolide), polyanhydride, polyorthoester, polyetherester, polycaprolactone, polyesteramide, poly(butyric acid), poly(valeric acid), polyurethane, polyacrylate, ethylene-vinyl acetate copolymers, acrylic-substituted cellulose acetate, non-degradable polyurethane, polystyrene, polyvinyl chloride, polyvinyl fluoride, poly(vinyl imidazole), chlorosulphonate, polyolefins, polyethylene oxide, polyvinylpyrrolidone (PVP), polyethylene glycol (PEG), polymethacrylate, hydroxypropyl methylcellulose (HPMC), ethyl cellulose (EC), hydroxypropyl cellulose (HPC), carboxymethyl cellulose, cyclodextrin, and copolymers of monomers forming the polymers.

20. The method according to claim 10, wherein the liquid formulation is mixed with an active ingredient, the active ingredient comprising at least one of a protein/peptide medicine, a hormone, a hormone analogue, an enzyme, an enzyme inhibitor, a signal transduction protein or a portion thereof, an antibody or a portion thereof, a single-chain antibody, a binding protein or a binding domain thereof, an antigen, an adherent protein, a structural protein, a regulatory protein, a toxin protein, a cytokine, a transcription regulator, a blood coagulation factor, and a vaccine.

21. The method according to claim 20, wherein the protein/peptide medicine comprises at least one of insulin, insulin-like growth factor 1 (IGF-1), growth hormone, erythropoietin, G-granulocyte-colony stimulating factors (CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormone-releasing hormone (LHRH), nafarelin, parathyroid hormone, pramlintide, enfuvirtide (T-20), thymalfasin, and ziconotide.

22. The method according to claim 18, wherein the solvent comprises inorganic or organic solvents comprising distilled water (DI water), methanol, ehanol, chloroform dibutyl phthalate, dimethyl phthalate, ethyl lactate, glycerin), isopropyl alcohol, lactic acid, and propylene glycol.
